(19) United States
(12) Patent Application Publication
(10) Pub. No.: US 2016/0250136 A1
(43) Pub. Date: Sep. 1, 2016

(54) UV CURABLE NAIL COATING COMPOSITIONS

(71) Applicant: AKCROS CHEMICALS LIMITED, Stratford Upon Avon, Warwickshire (GB)

(72) Inventors: Ian BRYSON, Edinburgh (GB); David THOMSON, Edinburgh (GB); Jiaqin LI, Bath (GB); Jemma BRYSON, Edinburgh (GB)

(21) Appl. No.: 15/027,102

(22) PCT Filed: Oct. 2, 2014
(86) PCT No.: PCT/GB2014/052980

§ 371 Date: Apr. 4, 2016

(30) Foreign Application Priority Data
Oct. 4, 2013 (GB) 1317635.9

(51) Int. Cl. A61K 8/84; A61Q 3/02; A61K 8/92

(52) U.S. Cl. CPC A61K 8/84; A61K 8/922; A61Q 3/02; A61K 2800/43

(57) ABSTRACT

This invention relates to the use of cationically UV curable compositions to coat nails, cationically UV curable nail coating compositions and methods of making up nails using said compositions. In a preferred embodiment, the cationically UV curable nail coating compositions comprise at least one epoxy resin. The epoxy resin may be of plant origin.

### FIELD

This invention relates to the use of cationically UV curable compositions to coat nails, cationically UV curable nail coating compositions and methods of making up nails using said compositions. In a preferred embodiment, the cationically UV curable nail coating compositions comprise at least one epoxy resin. The epoxy resin may be of plant origin.

### BACKGROUND

Ultra-violet (UV) radiation curable nail polishes which are based on acrylate/methacrylate-containing compositions have become established in the cosmetics market. This is due, in general, to the superior performance of the cured composition with respect to chipping and breaking, over more traditional nail polishes based on nitrocellulose dissolved in a solvent and which form a hard coating by solvent evaporation.

Current UV curable nail polishes are based on acrylate/methacrylate chemistries. The formulation typically comprises reactive monomers, oligomers, cross-linkers, photoinitiators, inhibitors, solvents and, for coloured layers, pigments. Several of these components can cause damage to skin and/or eyes. Some of the components may cause allergic reaction on skin contact. There is a need to provide a nail polish which has similar resistance to chipping and breaking properties as the current UV cured nail polishes and which also alleviates these problems.

Further, there is a need to provide a nail polish which is environmentally friendly, i.e., is at least partially derived from sustainable resources.

The painting or making up of nails using known UV curable nail polish is achieved by painting the liquid polymerisable nail polish formulation onto the nails which are then exposed to a UV light which causes the liquid formulation to cure to a dry solid film. Typically, the application of several layers of polish is involved in the making up of the nails. Generally a colourless coat is first applied which adheres well to the nail and which provides a good base onto which one or more coloured layers are then applied. A top coat of clear varnish is then applied to give a high gloss finish. Typically, all of the coats are UV curable coats. The UV curable coats must be cured after they have been applied, i.e. before the application of a subsequent layer. Notably the curing process leaves a sticky or tacky residue which needs to be wiped off using an alcohol wipe after the curing of the uppermost cured layer.

Acrylate/methacrylate chemistries cure by a chemical process that involves free radicals and this process has the disadvantage, particularly in the context of nail coatings, that the free radicals also react readily with oxygen and become unavailable for curing the (meth)acrylate. The curing process is said to be inhibited by oxygen. Oxygen in the air is sufficient to cause this inhibition and attempts to achieve a cured film in the presence of air result in a significant amount of uncured liquid formulation remaining on the surface of the nail where it is in contact with air. This is the sticky or tacky residue referred to above. The failure of the polish to cure fully has several important disadvantages for the application and wearing of nail polishes based on this chemistry.

Firstly, the uncured material has to be removed by wiping the nail coating with a cloth or pad wetted with a solvent, usually an alcohol such as isopropyl alcohol. This wiping of the nail coating with a solvent reduces the gloss finish of the coating. This is not a desirable effect for the topmost coat of polish. The wiping process requires the use of environmentally harmful solvents. It would be desirable to avoid the use of such solvents. Further, the wiping process is an additional chemical treatment step in the process of making up the nail, thus lengthening the process of making up the nail and this represents additional cost to the salons where nail polish is being applied.

In addition, the lack of complete curing is a problem because it persists throughout the thickness of the layer, not just at the surface, with the lower layers being more completely cured than the surface layers. Therefore, even after wiping, the cured nail coating still contains a residue of uncured formulation. As the finished layer is not fully cured the possibility exists that uncured monomers or other components in the coating can migrate to the surface of the film and eventually come into contact with the skin or eyes of the wearer. It is well known that some wearers experience skin irritation through touching their faces with their hands, the skin on the face being particularly sensitive. In extreme cases this can cause the skin of the wearer to become sensitised to the nail polish making further wearing of the nail polish impractical.

Another disadvantage associated with the current UV cured nail coatings is that of shrinkage. Shrinkage is an inherent phenomenon in the polymerisation and occurs when the liquid monomer molecules move closer together as they bond to form polymers. This has the consequence that the bond between the coating and the substrate to which it is applied is put under strain increasing the probability that the bond will fail. This is particularly important where the substrate is uncoated.

A popular trend in cosmetic nail coverings is that of surface decorations such as glitter, studs, crystals, gems and rhinestones to nails covered in nail polish. These decorations are generally glued onto the nail coating, i.e. in the case of UV cured compositions subsequent to the final curing step, and then dried in air. There is a need for a more efficient process for making up nails with nail polish and surface decorations. There is a tendency for the surface decorations to become dislodged and come off the coated nail surface. There is a need for a method of making up a nail with nail polish and surface decorations whereby the surface decorations are easily and securely attached.

There remains a need for nail coating compositions and methods of coating nails which alleviate some or all of these problems.

### SUMMARY OF THE INVENTION

The present invention is based, in part, on the finding that cationically UV curable compositions are particularly suitable for coating nails. In particular, it has been found that the use of cationically UV curable compositions in this new context, i.e. as nail coating compositions, can alleviate the aforementioned problems associated with known nail varnishes.

A first aspect of the invention relates to the use of a cationically UV curable composition as a nail coating composition. In a related aspect there is provided a method of coating a nail comprising applying a layer of a cationically UV curable composition to a nail and curing said layer using UV radiation. In a preferred embodiment, the cationically UV curable composition comprises at least one epoxy resin. The invention also provides a nail coating composition comprising at least one epoxy resin and a cationic photoinitiator. Preferably the composition comprises an epoxy resin of plant origin. The invention also provides for said nail coating composition in a cured state, and for layered nail coating systems and methods of making up nails based on said layered nail coating systems.

As used herein the term nail includes a human nail, animal nail, hoof, claw, a keratinous layer, an artificial nail and artificial nail tip. The term nail includes a coated nail. Thus the nail may be uncoated and have a natural or original surface or it may be coated, for example, with a base coat of nail polish or a colour coat of nail polish.

As used herein the terms layer, coat and film are interchangeable. As used herein the expressions nail coating composition, nail polish and nail varnish are interchangeable.

As used herein, the expression nail coating composition means that the composition can be applied directly onto the surface of a nail and/or it can be applied directly onto the surface of a coated nail thereby forming a layer in a multi-layered coating system for a nail. Thus nail coating compositions disclosed herein can be used to form individual layers in a system of layers applied to the surface of a nail.

The uses, methods and the compositions as disclosed herein are cosmetic, i.e. they are non-therapeutic uses, methods and compositions.

### DETAILED DESCRIPTION

A first aspect of the invention relates to the use of a cationically UV curable composition as a nail coating composition. By cationically UV curable is meant that the composition is capable of undergoing cationic polymerisation when exposed to UV radiation. This is a new use for cationically UV curable compositions. The inventors have found that using cationic curing chemistry in the coating of nails provides advantages over known nail coating technologies and, in particular, over known methods of coating nails using curable (meth)acrylate-based nail varnishes.

The cationic curing mechanism is different from the radical curing mechanism used in known UV curable (meth)acrylate-based nail varnishes. Surprisingly, it has been found that it is advantageous to apply this curing mechanism and associated curable materials to nail varnishes. The three most important differences that are significant for the application of these materials as nail polishes are:
1) the cationic curing process is not inhibited by oxygen and so the surface layers cure to a dry film requiring no “wipe off” to remove unreacted monomer;
2) unlike the radical curing mechanism, in which cross-linking takes place only while the curing film is exposed to the UV light, the cationic curing process continues after removal from the UV light thus enabling more complete curing to take place; and
3) the cationic curing process occurs in areas adjacent to but not directly illuminated by the UV light thus allowing areas not properly illuminated nevertheless to cure completely.

The better and more complete curing achieved by the cationic mechanism results in films or layers from which there is much less unreacted material to leach and which are also more resistant to having unreacted material leached out.

Of note is the fact that the cationic curing nail coating process requires no “wipe off” step. Thus the process is more efficient than the nail coating process for known UV curable (meth)acrylate-based nail varnishes; it take up less time and requires less materials (for example, there is no need for alcohol wipes). Another advantage of the complete curing and high gloss achievable with cationically cured formulations is the ability to achieve a high quality nail coating, which may be pigmented or clear and which may require only a single coating of the curable formulation to achieve the desired effect. Thus when the nail coating is a colour coat, no subsequent gloss coat is required. The nail coating compositions of the invention are highly durable when cured. Also, advantageously, the resultant cured nail coating suffers less shrinkage than conventional cured acrylate-based polymeric nail coating and thus can have better adhesive properties. Further, as the resultant cured nail coating contains less uncured components, when worn, there is in less transfer of uncured components from the wearer's nail to skin on the face, for example, where it can cause a problem.

The invention employs a cationically UV curable composition which is suitable for coating nails. In a preferred embodiment, the cationically UV curable composition comprises at least one epoxy resin.

### Epoxy Resin

By epoxy resin is meant a polymerisable material comprising one or more polymerisable compounds, each of which contains at least two epoxide functional groups. An epoxy resin thus comprises one or more monomers, dimers, oligomers and/or prepolymers containing at least two epoxide functional groups. By prepolymer is meant monomers, dimers or oligomers that have been reacted to an intermediate molecular weight state which is capable of further polymerisation to a fully cured state. The epoxy resin must be capable of cross linking to form a cured film and therefore must comprise polymerisable compounds having at least two epoxide functional groups. The epoxide functional group is represented by the formula:

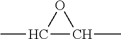

The epoxy resins useful in the invention include compounds comprising a molecular chain or molecular backbone with terminal and/or pendant epoxide groups. Typically, two terminal epoxide groups are present. The nature of the molecular chain can confer different properties to the cured films. The molecular chain can be aliphatic (non-cyclic), cycloaliphatic or aromatic, giving aliphatic, cycloaliphatic and aromatic epoxy resins, respectively. In each case the chain may have additional substituents, for example —OH groups, attached, provided that these are not amine groups or other groups with basic functionality that will inhibit cationic curing. The cationic curing mechanism is initiated by the formation of a protonic acid when UV light irradiates the photoinitiator. It is understood therefore that the epoxy resins used in the invention and cationically UV curable compositions of the invention exclude those that contain basic materials such as amines and urethanes, for example, epoxides with amine functionality, which would react with the protonic acid and inhibit the curing mechanism.

The at least one epoxy resin can be chosen from aliphatic epoxy resins, cycloaliphatic epoxy resins and/or aromatic epoxy resins.

Examples of aliphatic epoxy resins include: epoxide-containing fatty acid esters, alkyl diepoxides, epoxy-siloxanes, epoxy-polyesters, organic copolymers (such as vinyl compolymers) containing glycidyl ethers as comonomer and epoxidised alpha-olefins.

Examples of cycloaliphatic epoxy resins include: 3,4-epoxycyclohexylmethyl-3,4-epoxycyclohexanecarboxylate, bis-(3,4-epoxycyclohexyl)adipate, 1,2-epoxy-4-(epoxyethyl)cyclonexane, 2-(3,4-epoxycyclohexyl-5,5-spiro-3,4-epoxy)cyclohexane-metha-dioxane and also epoxycyclohexylalkyl dicarboxylates such as, for example, bis(3,4-epoxycyclohexylmethyl) pimelate, bis(3,4-epoxy-6-methylcyclohexylmethyl)maleate, bis-(3,4-epoxy-6-methylcyclohexylmethyl) succinate, bis-(3,4-epoxycyclohexylmethyl) oxalate, bis(3,4-epoxy-6-methylcyclohexylmethyl)sebacate, bis(3,4-epoxy-6-methylcyclohexylmethyl)adipate and limonene dioxide.

Examples of aromatic epoxy resins include: Bisphenol A diglycidyl ether, epoxy resins formed by the reaction of isopropylidenediphenol and epichlorohydrin, epoxy resins formed by the reaction of phenol-formaldehyde resins and epichlorohydrin.

In one embodiment, the epoxy resin comprises an aliphatic epoxy resin. Preferably the aliphatic epoxy resin comprises at least one epoxide-containing fatty acid ester. The ester may be synthetic or of animal or plant origin.

The ester group of the epoxide-containing fatty acid ester may be a glyceride, diglyceride, triglyceride, alkyl ester or allyl ester group. Preferably the ester is a triglyceride. It can be a mixed triglyceride (equivalent to one molecule of glycerol combined with two or three different fatty acids) or a simple triglyceride (equivalent to one molecule of glycerol combined with one fatty acid). When the epoxide-containing fatty acid ester is a triglyceride, preferably, for optimal film forming properties, the epoxide-containing fatty acid ester comprises an average number of epoxide groups per molecule of at least 3, or at least 4, and up to 5 or 6 and preferably the epoxide-containing fatty acid triglyceride has one epoxide group per fatty acid carbon chain or not more than two epoxide groups per fatty acid carbon chain.

The epoxide-containing fatty acid ester may contain one or more fatty acid carbon chains having a length of from C8 to C22, preferably C12 to C20, most preferably C16 to C18. By fatty acid carbon chain length is meant the number of carbon atoms in R, where R is a hydrocarbyl group in a fatty acid ester represented by RCOOR¹ and R¹ represents the ester group, where epoxidation of the fatty acid ester would result in the epoxide-containing fatty acid in question. The fatty acid carbon chain(s) may be linear or branched.

Preferably the epoxide-containing fatty acid ester is of plant origin. By “of plant origin” is meant that the ester is derived from a plant, i.e. prepared from starting materials of plant origin. Such starting materials include plant oils, i.e. oils which can be extracted from plants by subjecting plants to processes such as pressing and filtering. In their raw state, many plant oils contain fatty acid components and these are predominantly triglyceride esters of different fatty acids, with smaller proportions of di- and monoglycerides and free fatty acids. The oils contain both mixed triglycerides and diglycerides, i.e. based upon more than one fatty acid, and simple triglycerides and diglycerides each based on a single fatty acid. Many of the fatty acid triglycerides found in plant oils have carbon-carbon double bonds that can readily be reacted to form the corresponding epoxides. In the present invention, the resulting epoxide-containing triglycerides can be used directly or can be transesterified to produce other esters with desired properties, e.g. reduced viscosity. Advantageously, plant oils are sustainable resource and they are generally harmless for cosmetic applications.

The epoxide-containing fatty acid ester may be an epoxide-containing fatty acid triglyceride of plant origin. In this embodiment, the epoxy resin can comprise epoxidised plant oil, wherein the plant oil comprises said fatty acid triglyceride(s). Procedures for epoxidising plant oils are well known in the art and can be achieved, for example, by treating the plant oil with hydrogen peroxide or with a peracid such as peracetic acid. Also, in this embodiment, the epoxy resin can comprise a plant oil which contains, in its natural state, epoxide-containing triglycerides, e.g. Vernonia oil. Advantageously, plant oils provide a readily available and sustainable source of starting materials for the epoxy resins used in the cationically UV curable compositions described herein.

In one embodiment, the epoxide-containing fatty acid ester is derived from a plant oil selected from but not limited to the following plant oils: Borage, Calendula, Camelina, Castor, Coconut, Cottonseed, Crambe, Echium, Hemp, Jatropha, Jojoba, Lequerella, Linseed, Lunaria, Meadowfoam, High Erucic Rape, Rape, Safflower, Sunflower, Soya, Tung, Vernonia and Walnut oil. In one embodiment, the plant oil is not Calendula or Tung oil. Preferably the plant oil is selected from Borage, Camelina, Castor, Coconut, Cottonseed, Crambe, Echium, Hemp, Jatropha, Jojoba, Lequerella, Linseed, Lunaria, Meadowfoam, High Erucic Rape, Rape, Safflower, Sunflower, Soya, Vernonia and Walnut oil. More preferably the plant oil is selected from: Borage, Camelina, Echium, Hemp, Linseed, Safflower, Sunflower, Soya, Vernonia and Walnut oil. Even more preferably the plant oil is linseed or soya oil.

It will be clear to those skilled in the art that the oil from these plants comprise triglycerides of mixtures of fatty acids including significant proportions of unsaturated fatty acids that can be epoxidised. These fatty acids include: cis-hexadec-9-enoic acid, (palmitoleic); cis-octadec-9-enoic acid, (oleic); 12-hydroxy-cis-9-octadecenoic acid, (ricinoleic); cis,cis-9,12-octadecadienoic acid, (linoleic); (+)-(125,13R)-epoxy-cis-9-octadecenoic acid, (vernolic); 9-hydroxy-10,12-octadecadienoic acid, (dimorphecolic); 12-hydroxy-9,15-octadecadienoic acid, (densipolic); all-cis-9,12,15-octadecatrienoic acid, (alpha linolenic); all-cis-6,9,12-octadecatrienoic acid, (gamma linolenic); 8E,10E,12Z-octadeca-8,10,12-trienoic acid, (calendic acid); 9E,11E,13Z-octadeca-9,11,13-trienoic acid, (eleostearic); cis-6,9,12,15-octadecatetraenoic acid, (stearidonic); cis-11-eicosenoic acid, (gondoic/eicosenoic/gadolenic); cis-11-hydroxyeicosenoic acid, (lesquerolic); 9-eicosenioc acid, (gadoleic); eicosadienoic acid; D-14-hydroxyeicos-cis-11,cis-17-dienoic acid; auricolic acid; (Z)-docos-13-enoic acid, (erucic); docosadienoic acid and (Z)-tetracos-15-enoic acid, (nervonic).

In one embodiment, the epoxide-containing fatty acid ester contains one or more fatty acid chains associated with one or more of the above-identified fatty acids and, preferably, one or more of oleic acid, linoleic acid, alpha linolenic acid and gamma linolenic acid.

Preferably the epoxide-containing fatty acid ester is derived from a plant oil which comprises fatty acid esters containing fatty acid chains associated with one or more of the above-identified fatty acids and, more preferably, one or more of oleic acid, linoleic acid, alpha linolenic acid and gamma linolenic acid.

Preferably the plant oil comprises: (i) greater than 15% alpha linolenic or gamma linolenic acid and at least 15% linoleic acid (e.g. Borage, Camelina, Echium, Hemp and Linseed oil); or (ii) greater than 50% linoleic acid and greater than 20% oleic acid (e.g. Soya and Walnut oil); (iii) greater than 70% linoleic acid and at least 10% oleic acid (e.g. Safflower and Sunflower oil), or (iv) greater than 70% vernolic acid (e.g. Vernonia oil), expressed in terms of the equivalent molar percentages of the free fatty acids. The composition of plant oils may be expressed in terms of the equivalent molar percentages of the free fatty acids, i.e. the molar percentages of the fatty acid units RCOO, where R is the saturated or unsaturated fatty acid carbon backbone, present in the oil.

The choice of plant oil is made by reference to the required properties of the final liquid formulation, particularly its rheological properties, as well as the properties of the cured film. The preferred epoxide-containing fatty acid esters and plant oils described above, when used in the cationically UV curable compositions described herein, generally impart better film forming properties to the compositions. The preferred plant oils for the cationically UV curable compositions are generally those which contain fatty acid triglcerides with fatty acid chains having 16 to 18 carbon atoms e.g. fatty acid chains derived from oleic, linoleic and linolenic (alpha and gamma) acids.

The epoxide-containing fatty acid ester may be a mono-alkyl ester. By mono-alkyl ester is meant that the epoxide-containing fatty acid ester has one alkyl ester group, as if derived from the reaction of a monofunctional alcohol (an alcohol with one OH group) and a monobasic fatty acid. In this embodiment the preferred alkyl ester groups of the mono-alkyl esters are C1 to C8 alkyl groups, more preferably C1 to C4 alkyl groups and even more preferably a methyl or an ethyl group. The alkyl groups can be saturated or unsaturated. Preferably, the mono-alkyl ester is an ester of an epoxide-containing fatty acid. More preferably, the mono-alkyl ester is an ester of an epoxide-containing fatty acid that is of plant origin. For example such an ester can be prepared by epoxidising a plant oil, thus forming epoxidised mono, di- and tri-glyceride fatty acid esters and subsequently subjecting same to a transesterfication reaction with, for example, methanol. Suitable plant oils are as described for triglycerides and fatty acid esters above. The transesterification reaction can be catalysed by a metal alkoxide such as sodium methoxide, for example. This results in a glycerol fraction and a methyl ester epoxidised fatty acid fraction. The methyl ester fraction can be separated out, leaving the desired alkyl ester fraction. In this embodiment the composition of the invention can comprise epoxidised plant oil that has been transesterified and comprises the at least one mono-alkyl ester of an epoxidised fatty acid. In another preferred embodiment, the epoxide-containing fatty acid ester is a mono-alkyl ester of a plant oil which contains, in its natural state, epoxide-containing triglycerides, e.g. the product of transesterifying Vernonia oil. To be cationically polymerisable, the epoxide-containing mono-alkyl ester needs to have at least two epoxide groups. These can be situated on the fatty acid chain and/or can be obtained by epoxidising unsaturated bonds in the mono-alkyl unsaturated ester group.

The epoxide-containing fatty acid ester may be a mono-allyl ester. By mono-allyl ester is meant that the epoxide-containing fatty acid ester has one allyl ester group, as if derived from the reaction of a monofunctional allyl alcohol (an allyl alcohol with one OH group) and a monobasic fatty acid. By allyl is meant contains the CH₂═CH—CH₂— group. Preferably, the mono-allyl ester is an ester of an epoxide-containing fatty acid. More preferably, the mono-allyl ester is an ester of an epoxide-containing fatty acid that is of plant origin. For example such an ester can be prepared by epoxidising a plant oil, thus forming epoxidised mono, di- and tri-glyceride fatty acid esters and subsequently subjecting same to a transesterfication reaction with allyl alcohol. Suitable plant oils are as described for triglycerides and fatty acid esters above. The allyl function increases the reactivity of the ester, including the possibility of the ester being epoxidised thus further increasing the ability of the ester to cross-link to a cured film. In another preferred embodiment, the epoxide-containing fatty acid ester is a mono-allyl ester is a plant oil which contains, in its natural state, epoxide-containing triglycerides, e.g. the product of transesterifying Vernonia oil. To be cationically polymerisable, the epoxide-containing mono-allyl ester needs to have at least two epoxide groups. These can be situated on the fatty acid chain and/or can be obtained by epoxidising unsaturated bonds in the mono-allyl ester group.

Thus, in one embodiment, the epoxide-containing fatty acid ester can be chosen from epoxide-containing fatty acid glycerides, diglycerides, triglycerides, monoalkyl esters and monoallyl esters wherein the epoxide-containing fatty acid ester has a fatty acid carbon chain length of from C8 to C22, preferably of from C16 to C18. Preferably the epoxide-containing fatty acid ester is a triglyceride, monoalkylester or monoallyl ester of plant origin.

The cationically UV curable composition may comprise two or more epoxy resins. For example, when the composition comprises an epoxy resin which is an epoxide-containing fatty acid ester of plant origin, it may be desirable to also include at least one other epoxy resin which is different, e.g. not an epoxide-containing fatty acid ester, to modify the properties of the cured film e.g. to make it more wear resistant. The at least one other epoxy resin can be, for example, a cycloaliphatic epoxy resin, such as 3,4-epoxycyclohexylmethyl-3,4-epoxycyclohexanecarboxylate, and/or an aromatic epoxy resin, such as Bisphenol A diglycidyl ether or its oligomers.

The invention further provides a cationically UV curable composition which comprises (a) at least one epoxy resin and (b) a one cationic photoinitiator. Optionally, the composition further comprises one or more of (c) a crosslinker, (d) a photosensitiser, and (e) other additives such as a pigment.

### (a) Epoxy Resin

The epoxy resins useful in the compositions of the invention are as described above. The total amount of epoxy resins contained in the composition is preferably from 30 to 95 wt %, more preferably 40 to 85 wt%, even more preferably 50 to 80 wt % and most preferably 55 to 75 wt % based on the total weight of the composition. The proportion of the epoxy resin component that comprises an epoxide-containing fatty acid ester is from 0 to 100 wt % based on the total amount of epoxy resins. The proportion of the epoxy resin that is not an epoxy-containing fatty acid ester is from 0 to 100 wt % based on the total amount of epoxy resins. Thus in one embodiment, when the composition comprises at least one epoxide-containing fatty acid ester and at least one other epoxy resin which is not an epoxide-containing fatty acid ester, e.g. a cycloaliphatic epoxide, the total amount of epoxide-containing fatty acid esters present is from 5 to 70 wt % and the total amount of other epoxy resins present is from 25 to 90 wt % based on the weight of the composition. Preferably, the ratio of the total amount of epoxide-containing fatty acid esters to epoxy resins which are not epoxide-containing fatty acid esters is in the range of 1:2 to 2:1 by weight. Preferably, the ratio of the total amount of plant oil derived epoxy resins to non-plant oil derived epoxy resins is in the range of 1:2 to 2:1 by weight.

### (b) Cationic Photoinitiator

The composition further comprises a cationic photoinitiator. Cationic photoinitiators for use in cationic polymerisation are well known. They include onium compounds that photolyze to form a superacid species when excited with ultraviolet radiation. In the photolysis process, free radicals are also formed, and therefore, both epoxides and ethylenically unsaturated compounds can be polymerized in a dual-cure process. Preferably the cationic photoinitiators belong to the family of salts referred to as oniums, for example iodonium, or sulphonium metallic salts. Examples of commercially available cationic photoinitiators are: Irgacure 250, iodonium salt, PF₆⁻ (BASF); Irgacure 270, polymeric sulphonium salt, PF₆⁻ (BASF); Cyracure UVI-6976, sulphonium salt, SbF₆⁻ (Dow Chemical); Cyracure UVI-6992, sulphonium salt, PF₆⁻ (Dow Chemical); Degacure K185, sulphonium salt, PF₆⁻ (Degussa AG, marketed by Sartomer Compnay Inc); SP-150, sulphonium salt, PF₆⁻ (Asahi Denka); and Omnicat 550, sulphonium salt, PF₆⁻ (IGM Resins)

The cationic photoinitiator can be present in an amount of 0.5 to 10 wt %, or 1 to 7 wt % based on the weight of the composition. Typically more photoinitiator will be required for compositions containing pigment than compositions not containing any pigment. When pigment is present in the composition, the coating layer is more opaque allowing less light to pass through the film than if the pigment was not present. Therefore in order to get the same curing effect for a composition comprising pigment, it is necessary to increase the amount of photoinitiator.

### (c) Crosslinker

The composition may further comprise a crosslinker. Cross linkers are reactive species included in the formulation to modify the speed and extent of polymerisation, usually with the objective of achieving rapid and complete curing. Where, as in the present case, the primary resin is an epoxy resin and curing is to initiated by UV, these are polyols or small molecule epoxides. Both of these react with the epoxide functional group and serve to increase the cross-link density in the cured film. Crosslinkers may also include small molecule epoxides such as limonene dioxide and glycerol diglycidylether. Examples of polyols include Boltorn H2004 (a hyperbranched polyol) and trimethylolpropane (CAS No. 3047-32-3) (both from Perstop AB).

### (d) Photosensitiser

The composition may further comprise a photosensitiser. Photosensitisers are also referred to in the art as photoaccelerators. To improve the activity of the photoinitiator, sensitising agents such as thioxanthone derivatives can be used. They serve to enhance the rate of the polymerisation reaction and can do this, for example, by broadening the spectral response of the photoinitiator. Examples of commercially available photoaccerators/ohotosensitisers are: DBA, Anthracure 1331 (Kawasaki Kasei Chemicals Ltd.); DEA, Anthracure 1101(Kawasaki Kasei Chemicals Ltd.); Speedcure DETX, 2,4-diethylthioxanthone, CAS No. 82799-44-8 (Lambson); and Speedcure ITX, 2-,4-isopropylthioxanthone, mixed isomers, CAS No. 75081-21-9 (Lambson).

Photoinitiator and photosensitiser combinations can be chosen so as to tailor the curing of the composition to a particular wavelength range of UV light and also to overcome the reduction in reactivity that can be caused by the presence of pigment particles in the formulation. Thus the composition can be tailored so that it cures with particular UV light sources.

The photosensitiser can be present in an amount of 0.1 to 3 wt %, or 0.5 to 1.0 or 1.5 wt % based on the weight of the composition.

### (e) Other Additives

The composition may also contain other additives such as surface control additives (e.g. Tego glide (Evonik Industries AG)), reactive diluents such as propylene carbonate (reacts in the polymerisation and is not free within the cured formulation), pigment and a pearlescent.

In one embodiment, the composition comprises pigment and thus can act as a colour coat. Where the film contains a pigment to produce a coloured coating, the gloss of the cured coloured film is much greater than for known acrylic formulations such that the coloured film does not require a clear top coat to be applied in order to achieve a high gloss finish. A top coat or gloss coat is a layer of nail coating composition that is usually applied over a conventional colour coat to create a barrier surface which is resistant to chipping and to provide a high gloss shine.

In one embodiment the composition does not contain any pigment and is clear or transparent when cured and thus can function as a top gloss coat. The compositions of the invention provide a high gloss finish and are very durable. Conventional solvent-based nail varnishes are expected to last only 2-3 days before requiring renewal. There are other grades that can last up to a week. The cationic UV curable compositions of the invention can last up to two weeks or more. Thus, by applying a clear coat according to the invention to a traditional solvent-based coloured coat, it is possible to extend the lifetime of the colour coat to that associated with the top coat. The benefit is that the manufacturers can use their existing solvent-based colour formulations without having to rework all the colours.

The compositions of the invention do not contain any non-reactive solvent. By this is meant they do not contain any solvent that will not react in the curing step.

In one embodiment, the composition does not contain any wax.

The compositions are free of volatile organic compounds (VOCs), unlike acrylate formulations which can contain up to 50% by weight of organic solvent such as isopropyl alcohol, ethyl acetate or butyl acetate. These organic solvents are highly flammable substances.

The composition should have a sufficient viscosity to enable it be applied easily to a nail. The composition can have a viscosity of at least 10 mPas or in the range of 20 mPas to 20 Pas. The composition can have a viscosity in the range of 50 or 150 mPas to 400 mPas or in the range of 200 mPas to 330 mPas. As used herein viscosity is measured at 25 ° C. by means of a Cone & Plate viscometer using a 40 Poise cone fitted 19.1 mm diameter and 2° angle rotating at 750 RPM, according to ASTM D4287-00 (Reapproved 2010). An example viscometer is the REL Model CPD2000.

It has been found that the cured composition can be more easily removed from a nail than prior art polishes. A further advantage of the cured epoxide film is that it can have a toughness and flexibility that allows it to be removed from the nail by peeling and without the use of a solvent. Thus, in some embodiments, the cured composition can simply be peeled off the nail. The cured composition can be removed from a nail by soaking the layer in a solvent. Preferably the solvent is a polar solvent. Preferably the solvent comprises isopropanol and/or acetone.

The invention provides a method of coating a nail using a cationically UV curable composition. The cationically UV curable compositions described herein may be used in the methods of the invention.

There is provided a method of coating a nail comprising the steps of applying a layer of a cationically UV curable composition to a nail and cationically curing said layer with UV radiation. A brush, such as a conventional nail varnish brush, can be used to apply the composition so that it forms a layer on the nail. The curing is achieved by exposing the layer to UV radiation for a suitable period of time. UV radiation is electromagnetic radiation within the range of approximately 4 to 400 nm. UV light sources are widely available, for example, in the form of fluorescent, mercury vapour, incandescent or LED (light emitting diode) lamps.

In a further aspect of the invention there is provided a cured nail composition obtainable by the methods of the invention as described herein. Advantageously, the resultant cured composition suffers low shrinkage and thus can have good adhesive properties. Further, as the resultant cured composition contains less uncured components, when worn, there is in less transfer of uncured components from the wearer's nail to skin on the face, for example. Taken together with the lower hazard of the raw materials used in the formulation, this greatly reduces any risk to the wearer of skin sensitisation or irritation.

The cationically UV curable compositions can be used to form one or more layers in a multi-layered coating system for a nail. In one embodiment, the method of the invention is carried out on a coated nail. The coated nail may be coated, for example, with a conventional base layer based on UV-curable (meth)acrylate compositions or a conventional colour layer.

By base layer is meant a layer of nail coating composition which binds effectively with an uncoated nail and has a top surface that is receptive to and will bind to a subsequent layer of nail polish. Base layers are designed to adhere both to the natural or uncoated nail and to a subsequent layer of nail polish. By colour layer is meant a layer of nail coating composition which comprises a pigment. Conventional base and colour layers include those based on compositions comprising nitrocellulose dissolved in a solvent and which cure by drying and those based on UV-curable (meth)acrylate compositions which cure via a radical mechanism upon exposure to UV light. Similarly, conventional top coats can be nitrocellulose-solvent based or UV-curable (meth)acrylate based.

The invention also provides a method of embellishing nails by adding surface decorations such as crystals, gems, rhinestones, glitter, studs, beads, pearls, or shapes. The decoration can be metallic or plastic. The method involves applying a layer of the cationically UV curable composition onto a nail, placing the decorations on the layer and curing the layer with the decorations on it with UV light. This method has the advantage that it results in the nail decorations being held very firmly and avoids the need to have an extra gluing step. It is thus a more efficient process of decorating a nail.

In one embodiment, the method involves applying a clear cationic UV curable composition to a coated nail and curing said layer with UV-light, wherein the coated nail is coated with a coating which is a pigment-containing or colour layer. The pigment-containing layer can be a conventional nitrocellulose-based colour layer or a UV-cured (meth)acrylate-based polymer layer. No wipe off step is needed after the cationic curing of the clear layer in contrast with conventional cured acrylate polymer layers. Furthermore, the cured coating composition has a high gloss. In this embodiment, the method of the invention provides a top gloss coat to conventional nail polishes. When the colour coat is a nitrocellulose-solvent based coat, the top gloss coat of the invention provides a chip resistant, durable gloss coating that greatly extends the lifetime of the underlying colour coat. When the colour coat is a UV cured (meth)acrylate-based nail polish, the method provides a more efficient method of providing a top gloss coat. Preferably the method also comprises a step of coating the nail with a conventional colour layer, or the steps of coating the nail with a conventional base layer and then applying a conventional colour coat, prior to applying the cationically UV curable composition.

In one embodiment, there is provided a method of coating a nail with a cationically UV curable composition wherein the cationically UV curable composition forms a clear layer when cured and wherein said nail is already coated with a conventional base layer based on a UV-curable (meth)acrylate composition. In this embodiment, the clear layer according to the invention either acts as a top gloss coat or can act as a bonding layer for subsequent epoxy-based UV-cured layers according to the invention. The method can further comprise a step of coating the nail with the conventional base layer, prior to applying the cationically UV curable composition.

In one embodiment, there is provided a method of coating a nail with a cationically UV curable composition wherein the cationically UV curable composition forms a colour layer when cured. The colour layer can be bonded to a conventional base coat by way of an intermediate bonding layer. The intermediate/bonding layer may be a cationic UV curable composition according to the invention which contains no pigment, as described above. Thus there is also provided a method of coating a nail wherein the cationically UV curable composition forms a colour layer when cured and wherein said nail is already coated with clear layer of cationic UV curable composition according to the invention. In this embodiment, a clear epoxy-based UV-cured layer according to the invention is already present on the nail. This layer enables the bonding of the coloured epoxy-based UV-cured layer to the base coat. In this embodiment, advantageously, no top gloss coat is required. The epoxy-based UV-cured layer has a high gloss and is chip resistant and durable. The method can further comprise a step of coating the nail with a conventional base layer, or the steps of coating the nail with a conventional base layer, then coating the nail with a clear cationically UV curable composition, prior to coating the nail with the colour cationically UV curable composition.

There is also provided a layered coating system which contains at least one layer according to the invention. In one embodiment, there is provided a layered coating system comprising a conventional base layer based on a UV-curable (meth)acrylate composition, a cured clear layer according to the invention (acting as a bonding layer) and a cured colour layer according to the invention. In another embodiment, there is provided a layered coating system comprising a conventional base layer, a conventional colour layer and a cured clear layer according to the invention. The invention provides for layered nail coating systems obtainable by the methods of coating nails disclosed herein.

As used herein the term “comprising” encompasses the terms “consisting essentially of” and “consisting”.

The invention will now be described by way of the following non-limiting examples.

### EXAMPLES

### Examples 1 and 2

Nail coating compositions according to the invention, with the formulations shown in Table 1, were prepared by mixing the components shown in Table 1. The numerical values shown for the amounts of components are based on % by weight of active raw materials.

TABLE 1

| Component | Example 1 | Example 2 |
|---|---|---|
| Fully epoxidised soya (CAS No. 8013-07-8) | 35.75 | 33.4 |
| 3,4 ECC; 3,4-epoxycyclohexylmethyl 3,4- | 35.5 | 29.8 |
| epoxycyclohexanecarboxylate | | |
| (CAS No. 2386-87-0) | | |
| Boltorn H2004; Dendritic polyol | 10 | 6.8 |
| (CAS No. 462113-22-0) | | |
| Tego Glide 435 | 1 | 0.5 |
| Irgacure 270 | 2.55 | 4 |
| Anthracure UVS-1331; | 0.75 | 0.75 |
| 9,10-dibutoxyanthracene | | |
| (CAS No. 76275-14-4) | | |
| Propylene carbonate (CAS No. 108-32-7) | 14.45 | 22.7 |
| Pigment Red 57.1 (C.I. 15850:1) | 0 | 1.45 |
| Pigment White 6 (C.I. 778910) | 0 | 0.6 |
| | Total = 100 | Total = 100 |

The resulting formulations had viscosities of 180 mPas for Example 1 and 20 mPas for Example 2. Viscosity was measured at 25° C. by means of a Cone & Plate viscometer using a 40 Poise cone fitted 19.1 mm diameter and 2° angle rotating at 750 RPM using a REL Model CPD2000 viscometer. Example 1 contains no pigment and provides a clear coat when cured. Example 2 is a colour nail coating composition.

### Examples 3 to 5

In these examples, nail coating compositions according to the invention were used to make up nails according to the layered nail coating combinations indicated in Table 2.

TABLE 2

| | Example 3 | Example 4 | Example 5 |
|---|---|---|---|
| Third | None | Clear coat | Colour coat |
| layer | | according to | according to |
| | | Example 1 | Example 2 |
| Second | Clear coat | Conventional | Clear coat |
| layer | according | UV cured | according to |
| | Example 1 | (meth)acrylate- | Example 1 |
| | | based colour | |
| | | coat*** | |
| First | Conventional | Conventional | Conventional |
| layer | nitrocellulose- | UV cured | UV cured |
| | based colour | (meth)acrylate- | (meth)acrylate- |
| | coat* | based base | based base |
| | | coat** | coat** |

*The conventional nitrocellulose-based colour coat used was Boot's No. 7 Stay Perfect, colour “Devil's Delight”.
**The conventional UV cured (meth)acrylate-based base coat used was SensatioNail Gel Base & Top Coat.
***The conventional UV cured (meth)acrylate-based colour coat used wasSensatioNail Gel Polish, colour “Scarlet Red”.

In these examples, a first layer was applied to an uncoated human nail, first layer was dried/cured, a second layer was applied to the first layer and was dried/ cured and then, when used, a third layer was applied to the second layer and dried/cured.

In Example 3, a conventional nitrocellulose-based colour coat was applied to an uncoated human nail. This was cured by air-drying. Then a layer of the composition according to Example 1 was applied to the dried colour layer and was cured using UV light. The resultant top coat had a high gloss (93 gloss units at 60° angle, all gloss measurements in these examples were measured using ASTM-E-284). Durability of the overall treatment was assessed in human nail trials over a period of 14 days. The degree of wear was compared to that of the same nitrocellulose first layer applied without the cationic uv cured top coat. The wearable life of the treatment (i.e. before the level of chipping required removal of or repair to the coating) with the cationic uv cured was determined as between 10 and 14 days compared to the nitrocellulose-only treatment which lasted three to five days.

In Example 4, a conventional UV curable (meth)acrylate-based base coat was applied to an uncoated human nail. This was cured using UV light and, a conventional UV cured (meth)acrylate-based colour coat was then applied to it. This was also cured using UV light and an alcohol wipe was used to wipe off any uncured material. Then a layer of the composition according to Example 1 was applied to the dried colour layer and was cured using UV light. The resultant top coat had a high gloss (93 gloss units at 60° angle). Durability was assessed as in Example 3 and found to be 10 to 14 days. For comparison, a nail treatment with the same base coat and colour coat as Example 4 but using a conventional UV cured (meth)acrylate clear top coat (SensatioNail Gel Base & Top Coat) had a gloss of 60 gloss units at 60° angle and a durability of 10 to 14 days.

In Example 5, a conventional UV curable (meth)acrylate base coat SensatioNail Gel Base & Top Coat) was applied directly to an uncoated nail. After alcohol wipe off, a layer according to Example 1 was applied and cured. A colour coat according to Example 2 was then applied and cured. The resultant top coat had a high gloss (95 gloss units at 60° angle). Durability was assessed as in Example 3 and found to be 10 to 14 days. For comparison, a formulation comprising only conventional UV cured (meth)acrylate layers (base coat;SensatioNail Gel Base & Top Coat: colour coat; SensatioNail Gel Polish, colour Scarlet Red: top coat; SensatioNail Gel Base & Top Coat) had a gloss of 60 gloss units at 60° angle and a durability of 10 to 14 days.

1. Use of a cationically UV curable composition as a nail coating composition.

2. A method of coating a nail comprising applying a layer of a cationically UV curable composition to the nail and cationically curing the layer using UV radiation.

3. A use according to claim 1 or method according to claim 2 wherein the cationically UV curable composition comprises at least one epoxy resin.

4. A use or method according to claim 3 wherein the epoxy resin comprises at least one epoxide-containing fatty acid ester.

5. A use or method according to claim 4 wherein the epoxide-containing fatty acid ester is of plant origin.

6. A cationically UV curable composition for coating nails which comprises at least one epoxy resin and a cationic photoinitiator.

7. A cationically UV curable composition according to claim 6 which has a viscosity of at least 10 mPa.s.

8. A cationically UV curable composition according to claim 6 or claim 7 which comprises a photosensitiser.

9. A cationically UV curable composition according to any one of claims 6 to 8 wherein the epoxy resin comprises at least one epoxide-containing fatty acid ester.

10. A cationically UV curable composition according to claim 9 wherein the epoxide-containing fatty acid ester is an epoxide-containing fatty acid triglyceride, monoalkyl ester or monoallyl ester.

11. A cationically UV curable composition according to claim 10 wherein the epoxide-containing fatty acid ester is an epoxide-containing fatty acid triglyceride having an average number of epoxide groups per triglyceride molecule of at least 3.

12. A cationically UV curable composition according to any one of claims 9 to 11 wherein the epoxide-containing fatty acid ester has one or more fatty acid carbon chains of length C8 to C22.

13. A cationically UV curable composition according to any one of claims 9 to 12 wherein the epoxide-containing fatty acid ester is of plant origin.

14. A cationically UV curable composition according to claim 13 wherein the epoxide-containing fatty acid ester is derived from a plant oil selected from Borage, Calendula, Camelina, Castor, Coconut, Cottonseed, Crambe, Echium, Hemp, Jatropha, Jojoba, Lequerella, Linseed, Lunaria, Meadowfoam, High Erucic Rape, Rape, Safflower, Sunflower, Soya, Tung, Vernonia and Walnut oil.

15. A cationically UV curable composition according to claim 13 wherein the epoxide-containing fatty acid ester contains one or more fatty acid chains associated with one or more of oleic acid, linoleic acid, alpha linolenic acid and gamma linolenic acid.

16. A cationically UV curable composition according to claim 13 wherein the epoxide-containing fatty acid ester is derived from a plant oil which comprises:
(i) greater than 15% alpha linolenic or gamma linolenic acid and at least 15% linoleic acid; (ii) greater than 50% linoleic acid and greater than 20% oleic acid; (iii) greater than 70% linoleic acid and at least 10% oleic acid or (iv) greater than 70% vernolic acid, expressed in terms of the equivalent molar percentages of the free fatty acids in said plant oil.

17. A cationically UV curable composition according to any one of claims 9 to 16 which comprises at least one further epoxy resin chosen from cycloaliphatic epoxides and aromatic epoxides.

18. A cationically UV curable composition according to claim 17 wherein the further epoxy resin is 3,4-epoxycyclohexylmethyl-3,4-epoxycyclohexanecarboxylate or Bisphenol A diglycidyl ether.

19. A cationically UV curable composition according to any one of claims 6 to 18 wherein the total amount of epoxy resin is from 30 wt% to 90 wt%, based on the total weight of the composition.

20. A cationically UV curable composition according to any one of claims 9 to 19 wherein the composition comprises at least one epoxide-containing fatty acid ester and the total amount of epoxide-containing fatty acid esters is from 5 to 70 wt %, based on total weight of the composition.

21. A cationically UV curable composition according to any one of claims 6 to 20 which is clear when cured.

22. A cationically UV curable composition according to any one of claims 6 to 20 which comprises a pigment.

23. A use according to any one of claims 1 or 3 to 5 or a method according to any one of claims 2 to 5 wherein said cationically UV curable composition is according to any one of claims 6 to 22.

24. A cured nail coating composition obtainable by the method of claim 23.

25. A use according to any one of claims 1, 3 to 5 and 23 or a method according to any one of claims 2 to 5 and 23 wherein said nail is a coated nail.

26. A method according to any one of claims 2 to 5, 23 and 25 which comprises applying a layer of said composition to the nail and placing nail decorations on said layer prior to curing the layer with UV radiation.

27. A method of coating a nail according to claim 23 wherein said cationically UV curable composition forms a clear layer when cured and is according to claim 21, and said coated nail is coated with either a nitrocellulose-based colour coat or a cured (meth)acrylate-based colour coat.

28. A method of coating a nail according to claim 27 which further comprises either (i) applying a nitrocellulose based colour layer to the nail and drying the layer, or (ii) applying a UV curable (meth)acrylate-based colour layer to the nail and curing the layer using UV radiation, prior to applying a clear layer according to the method of claim 23 when the cationically UV curable composition is according to claim 21.

29. A method of coating a nail according to claim 23 wherein said cationically UV curable composition forms a colour layer and is according to claim 22, and said coated nail is coated with a clear nail composition according to claim 21 which is cured.

30. A method of coating a nail according to claim 29 which further comprises applying a clear layer according to the method of claim 23 when the cationically UV curable composition is according to claim 21 prior to applying a colour layer according to the method of claim 23 when the cationically UV curable composition is according to 22.

31. A method of coating a nail according to claim 30 which further comprises applying a UV curable (meth)acrylate-based base layer to the nail and curing the layer using UV radiation prior to applying the clear layer.

32. A layered nail coating system comprising:
a cured (meth)acrylate-based base layer;
a cured clear layer according to claim 24 wherein the cationically UV curable composition is according to claim 21; and
a cured colour layer according to claim 24 wherein the cationically UV curable composition is according to claim 22.

33. A layered nail coating system comprising:
a nitrocellulose-based or cured (meth)acrylate-based colour layer; and
a cured clear layer according to claim 24 wherein the cationically UV curable composition is according to claim 21.
